(19) United States
(12) Patent Application Publication
(10) Pub. No.: US 2019/0125272 A1
(43) Pub. Date: May 2, 2019

(54) Using Helmholtz Minimum Free Energy Slopes to Define Glial Cells that Diagnose Brain Disorder

(71) Applicant: Harold Szu, Bethesda, MD (US)

(72) Inventor: Harold Szu, Bethesda, MD (US)

(21) Appl. No.: 15/975,455

(22) Filed: May 9, 2018

Related U.S. Application Data
(60) Provisional application No. 62/503,476, filed on May 9, 2017.

(51) Int. Cl. A61B 5/00; A61B 5/026; A61B 6/00; G06N 3/063; G06N 3/08

(52) U.S. Cl. CPC A61B 5/7267; A61B 5/0263; A61B 5/4082; A61B 5/4088; G06N 3/08; A61B 6/501; A61B 6/5217; G06N 3/063; A61B 5/4094

(57) ABSTRACT

A method of diagnosing a disorder includes obtaining a medical image of a subject. A Helmholtz Minimum Free Energy is computed from the medical image. A negative slope of the Helmholtz Minimum Free Energy is determined, from which a glial force is computed. The existence of a disorder in the subject is diagnosed if a value of the glial force is within a predetermined range. The disorder can be, for example, a brain disorder, such as Alzheimer's disease, Parkinson's disease, and/or schizophrenia. Other examples of disorders are epilepsy and rheumatoid arthritis. The subject can be, for example, a human subject.

### CROSS-REFERENCE TO RELATED APPLICATION

This is related to, and claims priority from, U.S. Provisional Application for Patent No. 62/503,476, which was filed on May 9, 2017, the entire disclosure of which is incorporated herein by this reference.

### FIELD OF THE INVENTION

The invention related to systems and methods of diagnosing and treating disorders, for example, brain disorders.

### BACKGROUND OF THE INVENTION

The human brain weighs about 3 pounds, and is made of gray matter (neurons) and white matter (fatty acid glial cells). Our brains consume about 20% of our entire body energy. As a result, many pounds of biological energy by-products, for example, beta Amyloids, are produced. In our brains, the billions of Astrocyte glial cells are silent partners, acting as servant cells to the billions of neurons, and are responsible, for example, for cleaning dead cells and energy production ruminants from those narrow corridors called the brain-blood barriers, as part of the glymphatic system. This phenomenon was discovered recently by M. Nedergaad & S. Goldman (“Brain Drain,” Sci. Am. March 2016). They discovered that a good quality sleep of about eight hours is important, or else professionals and seniors with sleep deficiencies will suffer from slow death dementia, for example, Alzheimer's disease (blockage at LTM at hippocampus or STM at frontal lobe); Furthermore, besides preforming the nighttime cleaning job, glial cells produce the Myelin sheath covering the nerve cells in the brain and spinal cord like a co-axial cable. When there exists a disorder, a person's own immune defense system might mistake the Myelin sheath as a viral protein and attack it; this de-myelinating disease is known as Multiple Sclerosis. The resulting short circuitries block motor control at the cerebellum, generating a crippling effect.

Because so many people are deficient in their sleeping habits, and are exposed to other causes of these and other brain disorders, these disorders affect a large percentage of the population, often showing only minor symptoms that gradually increase, negatively affecting quality of life and life expectancy. It is therefore crucial that such disorders be diagnosed and treated as early as possible.

### BRIEF SUMMARY OF THE INVENTION

According to an aspect of the invention, a method of diagnosing a disorder includes obtaining a medical image of a subject. There are several types of medical brain imaging, and each type has different useful characteristics. For example, X-ray imaging (CAT scan) is a shadow-casting gram defining the calcium bone skull or locating a brain tumor in thick tissue. Functional-Magnetic Resonance Imaging (f-MRI) makes use of hemodynamics in that the ions in red blood (hemoglobin cells) have a different magnetic frequency when the ions have been combined with oxygen (anti-ferromagnetic) or not (ferromagnetic). For example, in the later stages of a brain tumor, the cancer cells need no more oxygen (Warburg effect) and it grew very dense. The glia formula denominator has an average input dendrite tree distance Dⱼ≡Σₜ[W_{i,j}]Sₜ which when shrunk ΔDⱼ/Δt<0 becomes sub-millimeter in size, which should be taken as a serious warning sign. Computed Tomography (CT) is based on multiple directional weak X-ray illumination shadows casting digital scanning.

A Helmholtz Minimum Free (HMF) Energy is computed from the medical image. The HFE energy is only relatively defined up to a constant H_{brain}≡E_{brain}−TₒS_{brain}, where the constant will be cancelled by the gradient descent slope. A negative slope of the Helmholtz Minimum Free Energy is determined to be the attractive force, rather than repulsive force. A glial force is computed from this negative slope. The existence of a disorder in the subject is diagnosed if a value of the glial force is within a predetermined range; too strong implies too-dense neurons with narrow dendrite distance, indicating a dense tumor (due to the cancer Warburg effect of anaerobic energy production). Thus, the diagnosis based the glia formula is relatively tracking the abnormal change of glue force estimated by the density of dense tissue; the actual value will be determined by the consistency of the inverse integration of brain imaging (this might appear to be a tautology; however, one can relate measurable distance relative by observing weekly growth change rates):

$$HFE = {\sum }_{j}^{ } \int {g}_{j} {dD}_{j} = - {\sum }_{j} \int \frac{Δ {H}_{brain}}{Δ {D}_{j}} {dD}_{j} = {\sum }_{j} \int \int \frac{Δ {H}_{brain}}{Δ {D}_{j}} \frac{Δ {D}_{j}}{Δ t} {dD}_{j} d Δ t$$

The disorder can be, for example, a brain disorder, such as Alzheimer's disease, Parkinson's disease, schizophrenia, and/or multiple sclerosis. Other examples of disorders are epilepsy and rheumatoid arthritis.

The subject can be, for example, a human subject.

### BRIEF DESCRIPTION OF THE DRAWINGS

FIG. 1 shows visual cortex Brodmann areas of the human brain.

FIG. 2A shows brain activity during epileptic seizures.

FIG. 2B shows epileptic seizure neurons.

FIG. 3 is a table showing functional EEG macroscopically quantifying internal states of the brain.

FIG. 4 is a drawing illustrating retention of information in the brain.

FIG. 5 illustrated power of pairs input processing.

FIG. 6 is a diagram illustrating how to update a centroid.

FIG. 7 is a diagram demonstrating hippocampus associative memory.

FIG. 8 shows the use of artificial neural networks to reduce the false alarm rates.

FIG. 9 illustrates power-of -pairs agreement and disagreement to distinguish signal and noise.

FIG. 10 shows the physical and functional relation between neurons and glial cells.

FIG. 11 depicts the six types of glial cells.

FIG. 12 is a depiction of a census of neuron cell types.

FIG. 13 depicts genetic and epigenetic properties of a biological cell.

FIG. 14 depicts experimental evidence of action potential formation in dendrites.

FIG. 15 is an illustration of the flushing out of biological energy by-products from the brain by the glymphatic system.

FIG. 16 shows how classical ANN relates with modern BNN.

FIG. 17 shows deep learning back-prop mediated through glial cells in BNN.

### DETAILED DESCRIPTION OF THE INVENTION

The present invention includes a mathematical definition of glial cells responsible for computational Artificial Intelligence (AI) in medical diagnosis, especially for Tumor Nodes Metastasis (TNM). This computational methodology is called Unsupervised Deep Learning (UDL), “deep” in the sense of multiple layers for a convex classifier. The UDL theory is based on the thermodynamic equilibrium of human brains that are kept at a constant temperature Tₒ to make an effortless decision at the Minimum Free Energy (MFE). This is referred to as a Natural Intelligence (NI), in contrast to AI in the sense of a non-contrived straightforward decision. Likewise, the trustworthiness of an MFE classifier will be comprehensible by trace-back to Ortho-Normal (ON) and Salient Feature Vectors (SFV).

The MFE cost function is derived from first principles obtained from nature: one, the homeostasis principle; and two, real-time duplicative sensory inputs. The homeostasis condition maintains constant brain temperature, which implies constant biochemistry reaction rates resulting in the same learning experience among all generations of Homo-sapiens (that smart two-feet stand up human). The power of paired sensory inputs from eyes, ears, nostrils, tessellate tasting buds, tactile touching sensing has real-time pre-processing that exploits “the agreement is the signal, while the disagreement is the noise,” and the input signal energy relaxes to the averaged brain temperature Tₒ as the UDL. A mathematical definition of glial (Greek: glue) cells is thus implicated, of which modem knowledge in neuroscience seems to corroborate. There are tens of billions of neurons and hundreds of billions of glial cells that keep our brains operating smoothly.

Glioma brain tumors might be traced back genome or life style phenome, for example, nitride curing food preservatives, pesticides, constant radiation, and professional job exposure. Honorable John McCain, Arizona Senator, has notably suffered from Stage 1 glioma and had the tumor surgically removed, and now the metastasis of malignant cells has evolved to the terminating Stage 4 of glioma. This unfortunate fact could be traced back to his harsh six years of prisoner life during which he was tortured, fed with rotten cured food, and forced to endure sleep deprivation at the so-called “Hanoi Hilton,” during the Vietnam War.

According to the naming standard set by Ann Arbor, Duke, clinical (c) and pathology (p) for Tumor, Nodes, Metastasis (TNM) by both the Union for International Cancer Control (UICC) and American Joint Committee on Cancer (AJCC) combined into the United Nations World Health Organization, there are four stages of cancer cells: Carcinoma in situ, Intravasation, Extravasation, and Metastasis.

The main cancer growth may be traced back to the mutations in oncogenes and tumor suppressor genes, rather than the (Nobel Laureate Otto) Warburg effect, which is considered to be a result of these mutations. The Warburg effect may simply be a consequence of damage to the mitochondria (energy production organelles within our cells) in cancer, or an adaptation to low-oxygen environments within tumors, or a result of cancer genes shutting down the mitochondria, which are involved in the cell's apoptosis (program to death) that kills cancer cells. Because glycolysis provides most of the building blocks, despite the presence of oxygen, to proliferate the Warburg effect changes energy production from oxygen-related ATP reversible ADP to anaerobic fermentation is a metabolic process that consumes sugar in the absence of oxygen.

Shortfall: All of those descriptive and complex naming systems of TNM from Ann Arbor to Duke are useful in clinical diagnosis or by pathological usages in big three treatments (radioactive, chemical, and surgical). None of them can be easily applied by the Natural Intelligence (NI) computational approach.

Approach: To compute the glial cell formula, the numerator is the Helmholtz Minimum Free Energy (MFE) based on local temperature inflammation from three major (X-Ray scan, chemotherapy, biopsy surgical) medical imaging and then the glial force is computed from the negative slope of the MFE with respect to the dendrite net distance among cell clusters, which can proactively diagnose and improve early treatment of human brain disorders. The denominator is based on a tabulation of the shrinkage of dendrite net sizes due to an increase in the density of malignant cells, together with the local temperature elevation changing the stability of Helmholtz free energy.

Pathology: When the glial cell can no longer clean up the energy waste by-product peptides, beta Amyloids, the patient will suffer from dementia and Alzheimer's disease. Some genetic pre-disposer factors might lead to the epileptic seizure trembling, or schizophrenia. When myelin sheath fatty acid insulation coating has been mistaken as the virus protein and attacked by our own antibodies, the peeling off white matter can no longer function as the ion current insulation, resulting in ion leakage in the cerebellum connected to the spinal cord peripheral nervous system at the ankles, knees, and hips, known to be rheumatoid arthritis. It can also result in multiple sclerosis, crippling muscular control, an auto-immune disease.

### Deep Learning Back-Prop and Applications

Deep Learning is not a buzz word; but the word “deep” is necessary to biologically describe the human visual system (HVS) at the back of the head cortex 17 area, where multiple layers of neurons and glial cells function to extract salient features: colors, edges, shapes, texture, etc. for pattern recognition. Also mathematically speaking, a single layer of neurons and glial cells can separate a linear classifier at a different slope value, so that deep layers have multiple layers forming a convex hull classifier in order to minimize false alarm rates.
(i) The derivation of a deep learning back-prop algorithm for both biological Unsupervised Deep Learning (UDL) and classical Supervised Deep Learning (SDL) are given. While the cost function of UDL is Minimum Free Energy (MFE), the cost function of SDL is Least Mean Square (LMS) Errors between desired output and actual input. The pseudo-code is identical in both cost functions. The difference is the interpretation of error gradients that the negative MFE gradient defines the biological Neuroglia cells \vec{g}ⱼ; the negative LMS gradient defines the classical delta \vec{δ}ⱼ.
(ii) Albert Einstein well commented that science has a little to do with the truth, but more to do with consistency. Thus, he stressed further that “everything should be made as simple as possible, but not simpler.” This is how he integrated twice from the relativistic momentum change to force and to energy by dropping all the lower limits to reach the simple and elegant formula E=mC². Similarly, our mathematical modeling of Biological Neural Network (BNN) & Natural Intelligence (NI) should be as simple as possible but not any simpler. The information degree of freedom must be observed. It has been studied that the degree of freedom (d.o.f.) of ANN is limited to 13˜17% if one wishes to enjoy the fault tolerance (FT) of associative memory recall. This fact may be due to the requirement that the set of salient feature vectors (SFV) must be orthogonal to one another, so that the nearest neighbor within 45° of the SFV axis would be counted as it is identical. As a matter of fact, the human brain is the most underdeveloped territory on the Earth, because of the orthogonality requirement for the distinction, and also because of the maintaining of, instead of indoctrinated robotic machine the “free will” of the mankind for the joy of discovery and creativity.
(iii) The Human Visual System (HVS) begins with Deep Convolutional Learning for the ON sparse Feature Extraction (FE) at the back of head Cortex 17 area, for example layer V1 for color extraction; V2, edge; V3, contour; V4, Texture; V5-V6 etc. for the scale-invariant feature extraction for the survival of the species. Then, we follow the classifier in the associative memory Hippocampus called Machine Learning. The adjective “deep” refers to structured hierarchical learning at a higher level of abstraction with multiple layers of Convolution NNs to a broader class of machine learning to reduce a False Alarm Rate. It is necessary because of the nuisance False Positive Rate (FPR); but the detrimental False Negative Rate (FNR) could delay an early opportunity. Sometimes when one might be over-fitting in a subtle way, ANN becomes “brittle” outside the training set. (S. Ohlson: “Deep Learning: How the Mind overrides Experience,” Cambridge Univ. Press 2006.). Thus, BNN requires the growing, recruiting, and pruning/trimming of neurons to provide self-architectures.
(iv) The recent success of Big Data Analyses (BDA) by Internet Industrial Consortium can be leveraged in the context of the invention. For example, Google co-founder Sergey Brin sponsored and was surprised by the intuition, beauty, and communication skills displayed by the Al AlphaGo. As a matter of fact, the Google Brain AlphaGo Avatar beat Korean grandmaster Lee SeDol in the Chinese Go Game 4:1 as millions watched in real time Sunday Mar. 13, 2016 on the World Wide Web. This accomplishment surprised and surpassed the WWII Alan Turing definition of AI that cannot tell whether the other end is human or machine. Now six decades later, the other end can beat a human. Likewise, Facebook has trained 3-D color-block image recognition, and will eventually provide an age and emotional-independent facial recognition of up to 97%. YouTube will automatically produce summaries of all the videos in YouTube, and Andrew Ng at Baidu discovered the surprise result that the favorite pet of mankind is the cat, not the dog!
(v) As such, at the DARPA Information Innovation Office Mr. David Gunning demanded the reason for why the machine decision was cats, otherwise, the DoD could not trust machine decisions in order to execute adversary action. DARPA conducted a 5-year program from 2016-2021 to develop explainable AI (XAI). Examining deeper into deep learning technologies, which are more than just software: ANN and SDL, because the software has been with us over three decades, since 1988 developed concurrently by Paul Werbos (“Beyond Regression: New Tools for Prediction and Analyses” Ph. D. Harvard Univ. 1974), and McCelland, & Rumelhart (PDP, MIT Press, 1986). Notably, the key is due to the persistent vision of Geoffrey Hinton and his protegees: Andrew Ng, Yann LeCun, Yoshua Bengio, George Dahl, et al.(cf. Deep Learning, Nature, 2015), who have contributed major IT as scientists and engineers to a program on Massively Parallel , for example, Graphic Processor Units (GPU). A GPU has 8 CPUs per rack and 8×8=64 racks per noisy air-cooled room at a total cost of millions dollars. Thus, toward UDL, the process includes programming on a mini-supercomputer and then programming on the GPU hardware and changing the ANN software SDL to BNN “Wetware,” because the brain is a 3-D carbon-computing, rather than 2-D silicon computing, and therefore involves more than 70% water substance.

### Biological Neural Network

When Albert Einstein passed away in 1950, biologists wondered what made him smart and kept his brain for subsequent investigation for decades. They were surprised to find that his brain weighed about the same as an average human brain at 3 pounds, and by firing rate conductance measurement had the same number of neurons, about ten billion, as an average person. These facts suggested the hunt remains for the “missing half of Einstein's brain.” Due to the advent of brain imaging (f-MRI based on hemodynamics (based on oxygen utility of red blood cells to be ferromagnetic vs diamagnetic he combined with oxygen), Computed Tomography based, on multiple direction projection of micro-calcification of dead cells, Positron Emitting Tomography based on radioactive positron agents decay annihilated with electron and generated the internal X-rays), neurobiologists discovered the missing half of Einstein's brain to be the non-conducting glial cells (cells made mostly of fatty acids) that are smaller in size, about 1/10^{th}, of a neuron, but do all the work except for communication with ion firing rates. Now we known a brain takes two to tango: billions of neurons (gray matter) and a hundred billion glial cells (white matter). The missing half of Einstein's brain is the 100 B glial cells, which surround each axon as the white matter (fatty acids) that keep slow neuron transmit ions fast. The more (Oligodendrocytes Myelin Sheath) glial cells Einstein had, the faster Einstein's brain performed neuron communication. That is, if one can quickly explore all possible solutions, one will not make a stupid decision.

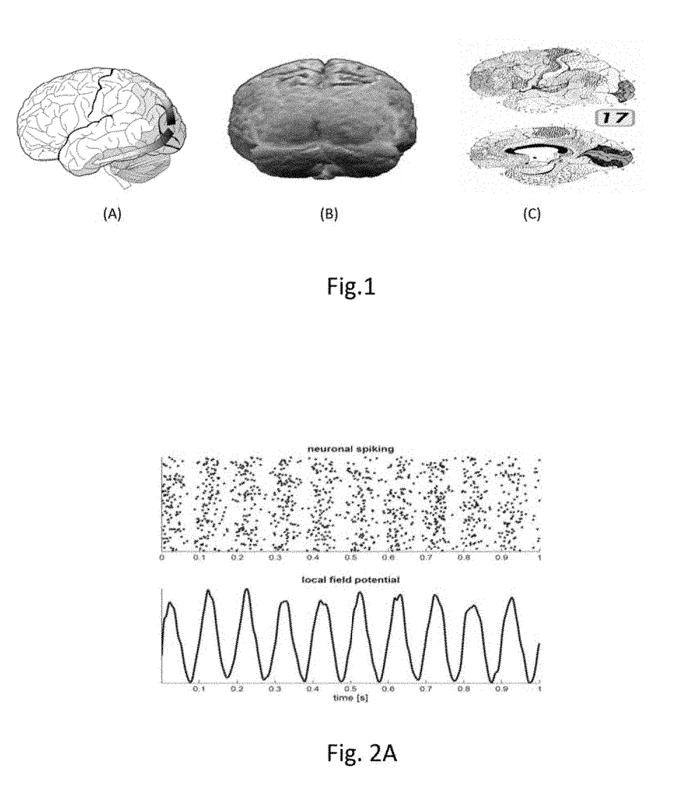

FIG. 1 shows Visual Cortex Brodmann Areas (BA) 17, 18, 19, and information flow paths (middle); the BA17 Occipital lobe has dorsal streams V1, V2, V5: where and how eyes and arms; and ventral streams V1, V2, V4 what LTM.

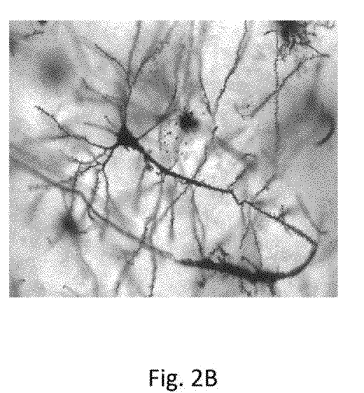

FIG. 2A shows epileptic seizures resulting from excessive feedback gain instability of neuronal feedback. A laser can burn off the feedback knot. FIG. 2B shows an epileptic seizure due to a short circuit cross-over knot with too-strong positive feedback. Laser burn-off of the knot is a treatment. Epileptic seizure may shed some light on firing spiking population and local field potential for the phase transition of the Helmholtz Free Energy (Szu et al SPIE News 2015) or a slow Alzheimer's dementia without Astrocytes neuroglial cells working hard during a good night's sleep.

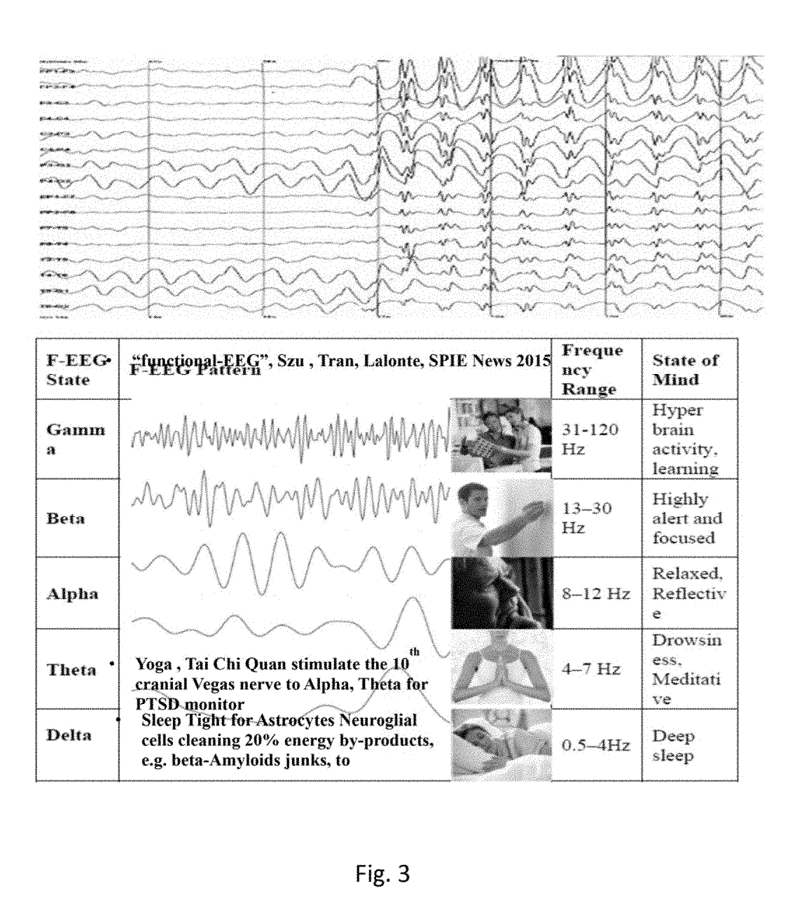

FIG. 3 is a table showing that functional EEG can macroscopically quantify the internal states of the brain.

Instead, the traditional approach of SDL is solely based on multiple layers of neurons as Processor Elements (PE) or Nodes of ANN. Instead of SDL training cost function the Least Mean Squares, using Least Mean Squares (LMS) Error Energy,

E=|(desired Output \vec{S}ₚₐᵢᵣₛ−actual Output Ŝₚₐᵢᵣₛ(t)|² (1)

### Sensory Unknown Inputs

Power of Pairs: \vec{X}ₚₐᵢᵣₛ(t)=[Aᵢⱼ]\vec{S}ₚₐᵢᵣₛ(t) (2)

where the agreed signals form the vector pair time series \vec{X}ₚₐᵢᵣₛ(t).

Uniformity of neuronal firing rate population may be measurable by the Boltzmann Entropy S. for a broader Natural Intelligence (NI). The internal state representation of the degree of uniformity of group of neurons' firing rates: \vec{S}ₚₐᵢᵣₛ(t) may be described with Ludwig Boltzmann entropy with unknown space-variant impulse response functions mixing matrix [Aᵢⱼ] and the inversion is determined by means of learning synaptic weight matrix.

Convolution Neural Networks: Ŝₚₐᵢᵣₛ(t)=[Wⱼᵢ(t)]\vec{X}ₚₐᵢᵣₛ(t) (3)

The unknown environmental mixing matrix is denoted [Aᵢⱼ]. The inverse is the space-variant Convolutional Neural Network weight matrix [Wⱼᵢ] of general type that can generate the internal states of knowledge representation.

Our unique and the only assumption, which is similar to early Hinton's Boltzmann Machine, is that the measure of degree of uniformity about the histogram or population of neuronal firing rates internal states is known as the entropy, introduced first by Ludwig Boltzmann.

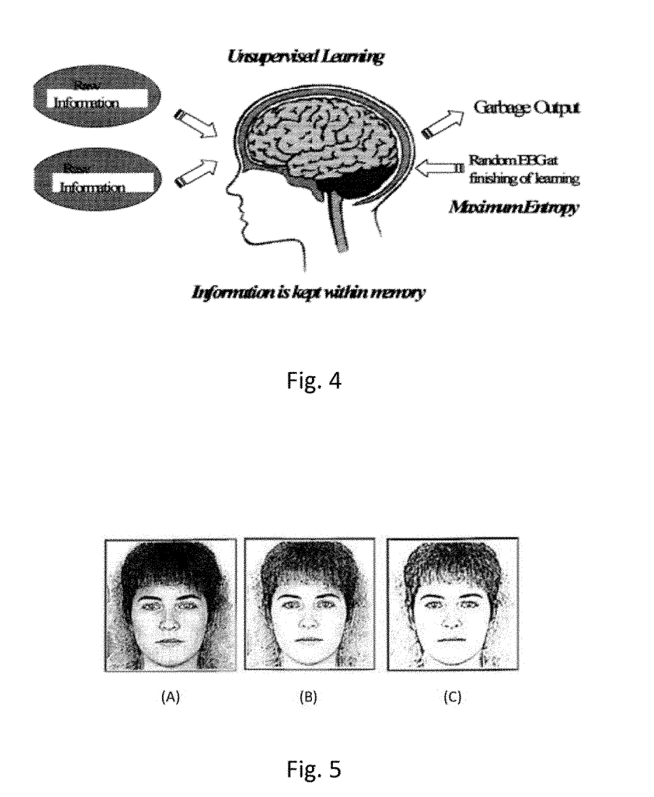

FIGS. 4 and 5A, B, C show how power of pairs keeps concurrent signals as information, filter out the disagreement as noise, and relax the local excitation into thermodynamic equilibrium. The information is kept in wavelets, or multiple resolution analysis (MRA).

### Introduction of ANN

ANN is massively parallel and distributed (MPD) (for example, a miniaturized Graphic Process Unit or software (for example, Python)) storage for the fault tolerant nearest-neighbor classifier. Beginning with the uniform average, one can recursively obtain a faster convergence by adding the difference between newcomer data with respect to the old averaged centroid. When Kalman generalized the uniform average with a weighted average, the constant numerical value became variable Kalman filtering. Furthermore, the weighted Kalman filtering is generalized with a “learnable recursive average” called the single layer of Artificial Neural Network, or Kohonen Self Organization Map (SOM), or Carpenter-Grossberg “follow the leader” Adaptive Resonance Theory (ART). This mathematics is relatively well known in early recursive signal processing. The new logic of ANN is augmented with a threshold logic at each processing elements (PE) or neuron nodes.

$$\begin{matrix} {\overline{x}}^{N} ≡ \frac{1}{N} {\sum }_{i = 1}^{N} {x}_{i} ⇒ {〈 x 〉}_{N} = \frac{1}{\sum {w}_{i}} {\sum }_{i = 1}^{N} {w}_{i} {x}_{i} ; & ( 4 ) \\ {\overline{x}}^{N + 1} ≡ \frac{1}{N + 1} {\sum }_{i = 1}^{N + 1} {x}_{i} = \frac{N + 1 - 1}{N + 1} \frac{1}{N} {\sum }_{i = 1}^{N} {x}_{i} + \frac{1}{N + 1} {x}_{N + 1} = {\overline{x}}^{N} + \frac{1}{N + 1} ( {x}_{N + 1} - {\overline{x}}^{N} ) & ( 5 ) \\ {〈 x 〉}_{N + 1} = {〈 x 〉}_{N} + K ( {x}_{N + 1} - {〈 x 〉}_{N} ) , & ( 6 ) \\ C = \frac{A + B}{2} = A + \frac{1}{2} ( B - A ) & ( 7 )\end{matrix}$$

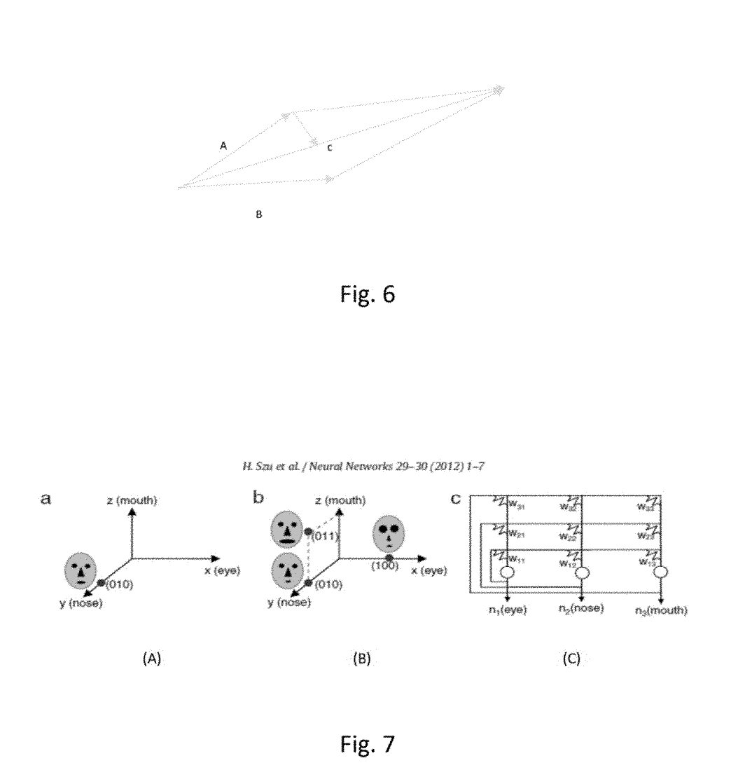

With reference to FIG. 6, there are two ways to update the centroid: The centroid vector \vec{C} may be computed in Eq. (7) from the old centroid vector \vec{A} and the new data \vec{B} that is different from the old centroid (\vec{B}−\vec{C}). This may be called following the leader \vec{A} becoming the new leader \vec{C} which is the centroid

$$\frac{\vec{A} + \vec{B}}{2} .$$

Furthermore, Artificial Neural Networks introduce the redundant outer and inner product at the Hippocampus Associative Memory [HAM].

This is why the mean average is replaced by adding the difference between the new input data with respect to the old averaged mean. It is in this spirit that Kalman has introduced the gain when the average is no longer the uniform average but a weighted average.

### Write by Outer Product:

[ ][ ]=[ ]=[HAM] (8)

### Read by Inner Product:

[HAM][ ]=[ ][ ]=[ ] (9)

Salient and orthogonal and normalized (ON) sparse features are extracted and then registered in multiple frames that will be less sensitive to the variations of direct pixel registrations. The ON nature will enjoy fault tolerance. For example, when a child is introduced to an uncle who has a big nose and an aunt who has big eyes, the child forms an ON salient Feature Extraction (FE) for big nose

$$[ \begin{matrix}0 \\ 1 \\ 0\end{matrix} ]$$

and big eyes

$$[ \begin{matrix}1 \\ 0 \\ 0\end{matrix} ]$$

FIG. 7 shows how Hippocampus Associative Memory (HAM) will be defined in a sparsely orthonormal feature.

$$[ HAM ] = [ \begin{matrix}0 \\ 1 \\ 0\end{matrix} ] [ 0 \; 1 \; 0 ] + [ \begin{matrix}1 \\ 0 \\ 0\end{matrix} ] [ 1 \; 0 \; 0 ] = [ \begin{matrix}0 & 0 & 0 \\ 0 & 1 & 0 \\ 0 & 0 & 0\end{matrix} ] + [ \begin{matrix}1 & 0 & 0 \\ 0 & 0 & 0 \\ 0 & 0 & 0\end{matrix} ] = [ \begin{matrix}1 & 0 & 0 \\ 0 & 1 & 0 \\ 0 & 0 & 0\end{matrix} ] \qquad ( 10 )$$

When big-nose uncle smiles, the feature will be

$$[ \begin{matrix}0 \\ 1 \\ 1\end{matrix} ]$$

and the question will be: “is he or isn't he?” Hippocampus Associative Memory (HAM) recall is the inner product

$$[ HAM ] = [ \begin{matrix}0 \\ 1 \\ 1\end{matrix} ] = [ \begin{matrix}1 & 0 & 0 \\ 0 & 1 & 0 \\ 0 & 0 & 0\end{matrix} ] [ \begin{matrix}0 \\ 1 \\ 1\end{matrix} ] = [ \begin{matrix}0 \\ 1 \\ 0\end{matrix} ] \\ yes , big nose uncle \qquad ( 11 )$$

This is why Homo sapiens require saliency by experience to prune those features that are irrelevant for survival. As such those ON FE can be Fault Tolerant (FT) for one-bit error 33% error tolerance; and abstraction and generalization are two sides of the same coin showing that laughing uncle is the same uncle as the NI.

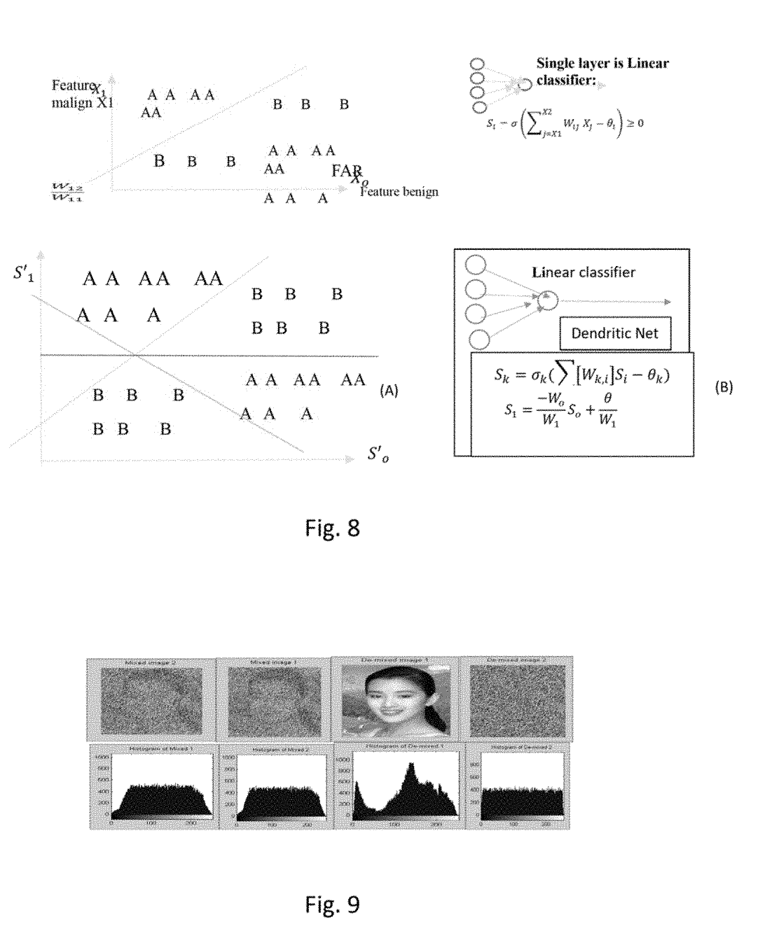

FIG. 8 shows Artificial Neural Networks (ANN) need multiple layers known as “Deep Learning” to reduce the False Alarm Rates (FAR). (A) The left panel shows that while a single layer of Artificial Neural Network can simply be a linear classifier shown in the Right Panel (B), multiple layers can improve the FAR denoted by symbol “A” included in the second class “B”. Obviously, it will take at least three linear classifier layers to completely separate both the mixed classes: A and B. When there are more than 2 salient features, one would need a lot more layers. That's why commercial MPD super computers have claimed nearly 100 layers of tens of thousands of nodes per layer in order to do computational intelligence.

### Theory of Natural Intelligence

Natural Intelligence (NI) is a kind of CI based on two necessary and sufficient principles observed from the common physiology of all animal brains (Szu et al., circa 1990).

Homeostasis Thermodynamic Principle: all animals roaming on the Earth have isothermal brains operated at a constant temperature Tₒ, (Homo sapiens 37° C. for the optimum elasticity of hemoglobin, chicken 40° C. for hatching eggs).

Power of Pairs: All isothermal brains have pairs of input sensors \vec{X}ₚₐᵢᵣₛfor the co-incidence account to de-noise: “agreed, the signal; disagreed, the noise,” for instantaneously processing.

FIG. 9 illustrates that the power of pairs indicate the agreed noisy image pixel can be separate those who are agreed as the signal image, while the disagreements are noise values.

Boltzmann defined the entropy to be a measure of the degree of uniformity, S=k log W.

(i) Total Entropy: Sₜₒₜ=k_{B} Log W_{MB} (12)

Solving Eq. (12) for the phase space volume W_{MB}, we derive the Maxwell-Boltzmann (MB) canonical probability for isothermal system.

$${W}_{MB} = exp ( \frac{{S}_{tot}}{{k}_{B}} ) = exp ( \frac{( {S}_{brain} + {S}_{env .} ) {T}_{o}}{{k}_{B} {T}_{o}} ) = exp ( \frac{{S}_{brain} {T}_{o} - {E}_{brain}}{{k}_{B} {T}_{o}} ) = exp ( - \frac{{H}_{brain}}{{k}_{B} {T}_{o}} ) \qquad ( 13 )$$

Use is made of the isothermal equilibrium of the brain in the heat reservoir at the homeostasis temperature Tₒ. Use is also used of the second law of conservation of energy ΔQₑₙᵥ.=TₒΔS_{env.} and the brain internal energy ΔE_{brain}+ΔQₑₙᵥ.=0, and then the change is integrated and the integration constant dropped due to arbitrary probability normalization. Because there are numerous neuron firing rates, the set of scalar entropy becomes the vector entropy for the representation of internal states for the degree of uniformity clusters of neuronal firing rates.

{Sⱼ}⇔\vec{S} (14)

Biologists might ask the reason why the entropy defined by Boltzmann is a proper measure of the degree of uniformity voting consensus of neuron firing rates population. Historically speaking, Boltzmann is survived only by his immortal formula. In 1912, Walter Nernst stated the 3rd law of thermodynamics: “It is impossible for any procedure to lead to the isotherm T=0 in a finite number of steps.” Because the Kelvin temperature can never reach absolute zero (given the ground state Higg's boson energy fluctuation), then incessant molecular collisions will mix toward maximum uniformity as the heat death as the Boltzmann basis of the irreversible increase of entropy toward the heat death. In other words, molecular collision will gradually erode the binging energy, the loss of archeology information dear to paleontologist at heart, for example, a landslide voting has maximum uniformity associated with no voter distribution information. Therefore, it is asserted that the physics entropy becomes an appropriate internal state of knowledge representation (ISKR). Boltzmann dis-information is Shannon information.

Henri Poincare observed keenly that all the dynamics both classical Newtonian and quantum mechanical is time reversible invariant (t⇔−t)

$${m}_{o} \frac{{d}^{2} \vec{X}}{{dt}^{2}} = {m}_{o} \frac{{d}^{2} \vec{X}}{{d ( - t )}^{2}} ; \pm i ℏ \frac{\partial Ψ}{\partial ( \pm t )} = - \frac{{ℏ}^{2}}{2 m} {∇}^{2} Ψ$$

We now know after all that Boltzmann is right, the trajectory is more than dynamics but initial boundary conditions which are time irreversible variant due to collision mixing.

ΔSₜₒₜ>0 (15)

We can assert the brain NI learning rule

ΔH_{brain}=ΔE_{brain}−TₒΔS_{brain}≤0. (16)

This is the NI cost function at MFE, useful in the most intuitive decision for Aided Target Recognition (AiTR) at Maximum PD and Minimum FNR for Darwinian natural selection survival reasons.

The survival NI is intuitively simple, flight or fight, using the parasympathetic nerve system as an auto-pilot.

Maxwell-Boltzmann equilibrium probability is derived early in Eq. (13) in terms of the exponential weighted Helmholtz Free Energy of the brain:

H_{brain}=E_{brain}−TₒS_{brain} (17)

### Toward BNN Morphology Architecture Learning

A brain logistic function is the normalization of two-state Maxwell-Boltzmann probability of connect or not as: ΔH_{brain}=H_{recruit}−Hₚᵣᵤₙₑ weighted by the homeostasis equilibrium

$$σ ( \frac{Δ {H}_{brain}}{{k}_{B} {T}_{o}} ) ≡ exp ( - \frac{{H}_{prune}}{{k}_{B} {T}_{o}} ) \text{/} \{ exp ( - \frac{{H}_{prune}}{{k}_{B} {T}_{o}} ) + exp ( - \frac{{H}_{recruit}}{{k}_{B} {T}_{o}} ) \} = 1 \text{/} [ 1 + exp ( - \frac{{H}_{prune}}{{k}_{B} {T}_{o}} ) ] = \{ \begin{matrix}1 , \frac{{H}_{prune}}{{k}_{B} {T}_{o}} ← ∞ \\ 0 , \frac{{H}_{prune}}{{k}_{B} {T}_{o}} ← - ∞\end{matrix} \qquad ( 18 a )$$

The slope of the brain sigmoid is merely a window function near the recruiting equilibrium

$$\frac{d σ ( {H}_{brain} )}{{dH}_{brain}} = σ ( {H}_{brain} ) \{ 1 - σ ( {H}_{brain} ) \} \qquad ( 18 b )$$

It is suggested that the positive growing brain will recruit new neurons (or prune old neurons that take too much energy to maintain) into a morphological changing brain (that will be demonstrated elsewhere). Note that Russian Mathematician G. Cybenko has proved “Approximation by Superposition of a Sigmoidal Functions,” Math. Control Signals Sys. (1989) 2: 303-314. Similarly, A. N. Kolmogorov, “On the representation of continuous functions of many variables by superposition of continuous function of one variable and addition, Dokl. Akad. Nauk, SSSR, 114 (1957), 953-956.

Homo sapiens at 37° C. (optimum for hemoglobin elasticity); while chicken 40° C. (for egg hatching); but chickens are lacking of an opposing big thumb for holding tools and becomes less intelligent than Homo sapiens (we eat them, not vice versa, Q.E.D.).

### The Lyapunov Stability Rule Implies Neurodynamics; Consequently, Hebb Learning Rule Implies Biological Glial Cells

Derivation of Newtonian equation of motion, the Biological Neural Networks (BNN) from the Russian Mathematician Aleksandr Mikhailovich Lyapunov, who has proved a monotonic absolute convergence theorem as follows: Since we have proved an equilibrium brain at MFE ΔH_{brain}≤0

$$\frac{Δ {H}_{brain}}{Δ t} = ( \frac{Δ {H}_{brain}}{Δ [ {W}_{i , j} ]} ) \frac{Δ [ {W}_{i , j} ]}{Δ t} = - \frac{Δ [ {W}_{i , j} ]}{Δ t} \frac{Δ [ {W}_{i , j} ]}{Δ t} = - {( \frac{Δ [ {W}_{i , j} ]}{Δ t} )}^{2} \leq 0 Q . E . D . \qquad ( 19 )$$

Therefore, Neurodynamics is merely the Newtonian equation of motion for the learning of a synaptic weight matrix, which follows from the brain equilibrium at minimum free energy (MFE) in the isothermal Hehnholtz sense

$$\frac{Δ [ {W}_{i , j} ]}{Δ t} = - \frac{Δ {H}_{brain}}{Δ [ {W}_{i , j} ]} \qquad ( 20 )$$

It takes two to tango. Unsupervised Learning becomes possible because BNN has both neurons as threshold logic and housekeeping glial cells as input and output.

Assume for the sake of the causality, the layers are hidden from outside direct input, except the 1^{st} layer, and the l-th layer can flow forward to the layer l+1, or backward, to l−1 layer, etc.

Defining the Dendrite Sum from all the firing rates \vec{S}ᵢ of the lower input layer represented by the output degree of uniformity entropy \vec{S}ᵢ as the following net dendrite vector:

\vec{Dendrite}ⱼ≡Σᵢ[W_{i,j}]\vec{S}ᵢ (21)

It is possible to obtain the learning rule observed by the co-firing of the presynaptic activity and the post-synaptic activity by Canadian neurophysiologist D. O. Hebb in 1949, namely, the product between the presynaptic glial input \vec{g}ⱼ and the postsynaptic output Firing Rate \vec{S}′ᵢ it is proved it directly as follows: glia were discovered in 1856, by the pathologist Rudolf Virchow in his search for a “connective tissue” in the brain; glial cell: a supportive cell in the central nervous system. Unlike neurons, glial cells do not conduct electrical impulses. The glial cells surround neurons and provide white matter glue support for and insulation between them. Glial cells are the most abundant cell types in the central nervous system, numbering about 100 billion. Six types of glial cells include oligodendrocytes, astrocytes, ependymal cells, Schwann cells, microglia, and satellite cells, which provide a unified theory of all, the axon output firing ions must be recruited from the synaptic gap matrix from the active house servant neuroglia cells connected from the dendrite other ends ions.

$$Neuroglia \text{:} \frac{Δ [ {W}_{i , j} ]}{Δ t} ≡ - \frac{Δ {H}_{brain}}{Δ [ {W}_{i , j} ]} = ( - \frac{Δ {H}_{brain}}{Δ \vec{{Dendrite}_{j}}} ) \frac{Δ \vec{{Dendrite}_{j}}}{Δ [ {W}_{i , j} ]} ≡ {\vec{g}}_{j} {\vec{S}}_{i} , \qquad ( 22 )$$

Following the Hebb rule of “wired together, fired together,” to produce the firing rate, there is no other choice but the rest must be housekeeping glial cells. Consequently,

Δ[W_{i,j}]=[W_{i,j}(t+1)]−[W_{i,j}(t)]=\vec{g}ⱼ\vec{S}ᵢη (23)

Where, in our brain η≈O|Δt|), the mathematical definition of glial cells follows:

$${g}_{j} ≡ - \frac{\partial {H}_{brain}}{\partial {Dendrite}_{j}} = - \frac{\partial {H}_{brain}}{\partial {S}_{j}} \frac{\partial {S}_{j}}{\partial {Dendrite}_{j}} = - \frac{\partial {H}_{brain}}{\partial {S}_{j}} {σ}_{j}^{′} ( {Dendrite}_{j} )$$

$$where - \frac{\partial {H}_{brain}}{\partial {S}_{j}} = - {\sum }_{k} \frac{\partial {H}_{brain}}{\partial {Dendrite}_{k}} \frac{\partial {Dendrite}_{k}}{\partial {S}_{j}} = - {\sum }_{k} \frac{\partial {H}_{brain}}{\partial {Dendrite}_{k}} \frac{\partial }{\partial {S}_{j}} {\sum }_{i} [ {W}_{k , i} ] {S}_{i} = - {\sum }_{k} \frac{\partial {H}_{brain}}{\partial {Dendrite}_{k}} [ {W}_{k , j} ] = {\sum }_{k} [ {W}_{k , j} ]$$

Denoting the next layer neuroglia cells with the tide superscript, then consequently UDL:

gⱼ=σⱼ(Dendriteⱼ){1−σⱼ(Dendriteⱼ)}Σₖ [symbol] [W_{k,j}]

This derives the multiple layer UDL:

[Wⱼᵢ(t+1)]−[Wⱼᵢ(t)]=\vec{g}ⱼ\vec{S}ᵢη=\vec{S}ᵢησⱼ(Dendriteⱼ){1−σⱼ(Dendriteⱼ)}Σₖ [symbol] [W_{k,j}]+αₘₒₘₜᵤₘ[Wⱼᵢ(t)−[Wⱼᵢ(t−1)]] (24)
Q.E.D.

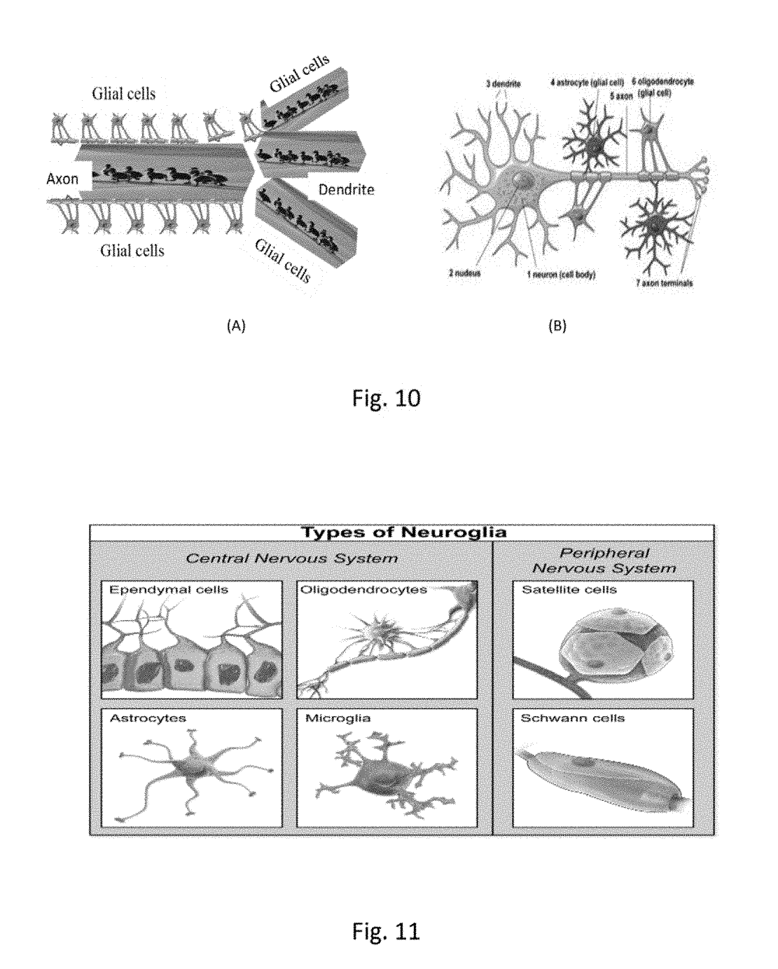

FIG. 10 shows ions about a thousand times larger than an electron that behave like large and slow ducks. Their transport through the axons is “like a row of ducks crossing the road.” Nevertheless, when one ion pushes in the other ion pops out in a pseudo-real time. The glial cells are fatty acids white matter in the brain that surround each axon output pipe to insulate the tube as coaxial tube. How can slow thermal positive-charge large ions that repel one another transmit along a meter-long axon cable from tail to toe? This is because the coaxial cable of the axon is surrounded by the insulating myelin sheath fatty acid oligodendrocytes glial cells. A autoimmune disease is one which the immune system attacks joints, or eats away at the protective covering of nerves, for example, rheumatoid arthritis pain or multiple sclerosis. Damaged nerve covering leads to nerve impulse disruption, for example, bladder dysfunction, bowel problems, crippling mobility and double vision. With normal nerve covering, “one ion pops in, the other ion pops out in a pseudo-real time.” The longest axon spans from the end of the spinal cord to the big toe which we can nevertheless control in real time running away from hunting lions.

Neuroglial biology insures four functionalities: (1) real time communication; (2) convex hull classifier; (3) multiple layer morphology with the help of multiple layer insulating glue glial cells; and (4) disorder might be implicated by the too strong glue divergence at the glial cells singularity.
(1) Real Time (RT) communication because axon ion vesicles are confined and aligned up in the axon cable surrounded by electrically insulated white matter myelin sheath glial cells, making the axon insulator an co-axial cable, becoming “how does the duck cross the road?” O(Δt)=10-th mille-sec. One meter long from the tail end of the spinal cord to the big toe running away for the survival of the species.
(2) Multiple layer convex hull classifier reduces the false alarm rate.
(3) Unified neuroglial theory by multiple dendrite morphologies insure multiple neuroglial.
(4) Divergence of the gradient may define the brain tumor glioma.

There are six kinds of glial cells (about one-tenth the size of neurons; four kinds in the CNS (astrocytes, microglia, ependymal, oligodendrocytes myelin sheath); two in the spinal cord: (satellite, schwann). They are more than silent partners and serve as house-keeping servant cells.

As shown in FIG. 11, functionally the glial cells surround each neuron axon output, in order to keep the slow neural transmission ions lined up inside the axon tube, so that one pushes in as the another pushes out in real time. In addition, they provide nutrients to the neuron.

R. Lipmann has introduced the momentum for classical ANN to go over a local minimum.

Sources of attractive field theory can be unified: electron radius, gravitational diameter, and to estimate that of glial cell size that varies from one of six kinds of glial cells.
1. Explicitly, the pre-synapse junction development depends on assistance from the glial cells for alternating the resting potential 75 mV for glutamine release. Glial cells growth factor deficiency may link to brain disorders such as schizophrenia
2. The Hodgkin-Huxley model, or conductance-based model, is a mathematical model that describes how action potentials in neurons are initiated and propagated, that approximates the electrical characteristics of excitable cells such as neurons and cardiac myocytes in 1952 to explain the ionic mechanisms underlying the initiation and propagation of action potentials in the squid's giant axon. They received the 1963 Nobel Prize in Physiology or Medicine for this work.
3. Gray Matter Neurons (William Herkewitz, Science Nov. 26, 2015)

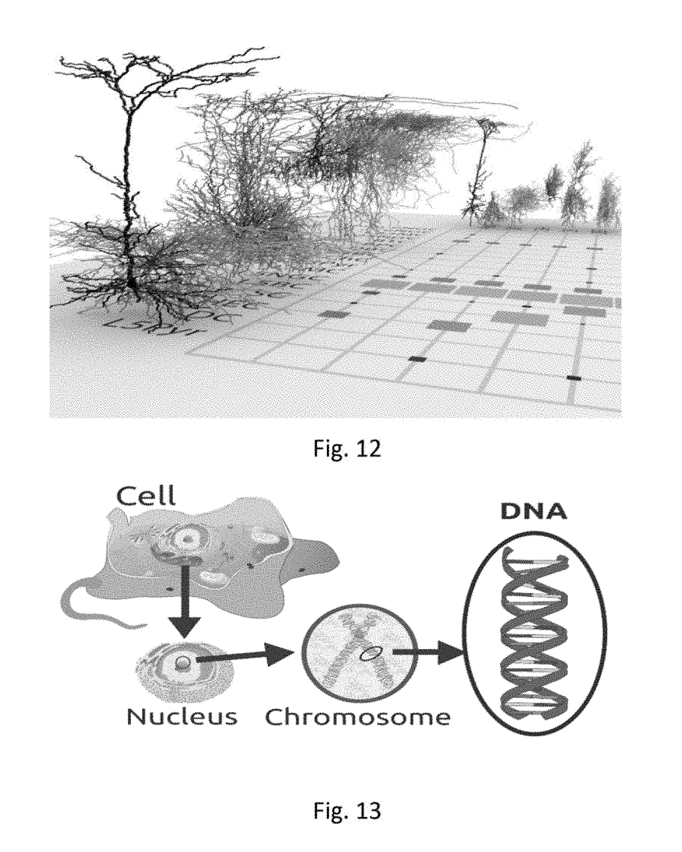

Referring to FIG. 12, Xiaolong Jiang and Andreas Tolias at Baylor College of Medicine in Houston announced six new types of 15 adult mice brain cells by the method of slicing razor-thin slices (RTS) of mature brain. “This RTS methodology has established a complete census of all neuron cell types is of great importance in moving the field of neuroscience forward,” says Tolias, at Baylor College of Medicine.

### Active Glial Cells Biology at Dendrite

Referring to FIG. 13, a biological cell has all genetic and epigenetic property. The idea that glial cells might have a role in learning seems contrary to the usual model of dendrite input soma summation action potential generation. Neurons are large, tree-like structures with extensive, branch-like dendrites spanning⇔1000 μm, but a small ˜10-μm soma.

For example: a classical electron radius

$$\frac{e}{r} e = E = {mC}^{2} ; r = \frac{{e}^{2}}{{mC}^{2}} = 2.8 \times {10}^{- 13} cm$$

$${g}_{j} = - \frac{Δ H}{Δ {D}_{j}} ; {D}_{j} = {\sum }_{k} [ {W}_{jk} ] {S}_{k} ;$$

$$\vert {g}_{j} \vert = 0.1 \vert Neuron \vert = \frac{\vert Δ H \vert}{\vert Δ {D}_{j} \vert} ;$$

$$\vert Δ H \vert = 0.1 \vert Neuron \vert \vert Δ {D}_{j} \vert$$

$$\vert Δ {D}_{j} \vert = 10 \frac{\vert Δ H \vert}{\vert neuron \vert}$$

It has recently been determined that active dendrites are about 100 times bigger (about 1000 μm) than soma cells about (10 μm), and so is the action potential, Moore et al. (Sci. 2017):
“Dendrites receive inputs from other neurons, and the electrical activity of dendrites determines synaptic connectivity, neural computations, and learning. The prevailing belief has been that dendrites are passive; they merely send synaptic currents to the soma, which integrates the inputs to generate an electrical impulse, called an action potential or somatic spike, thought to be the fundamental unit of neural computation. These ideas have not been directly tested because traditional electrodes, which puncture the dendrite to measure dendrite voltages in vitro, do not work in vivo due to constant movement of the animals that kills the punctured dendrites. Hence, the voltage dynamics of distal dendrites, constituting the vast majority of neural tissue, is unknown during natural behavior. Dendrites occupy more than 90% of neuronal tissue. However, it has not been possible to measure distal dendrite membrane potential and spiking in vivo over a long period of time. Moore et al. (Sci. 2017) developed a technique to record the subthreshold membrane potential and spikes from neocortical distal dendrites in freely behaving animals. These recordings were very stable, providing data from a single dendrite for up to 4 days. Unexpectedly, distal dendrites generated 100 times larger action potentials whose firing rate was nearly five times greater than at the cell body. Further Glial cell's with their insulating properties, suggest dynamics with a long time constant. This article (Moore, 2017), however, Neural activity in vivo is primarily measured using extracellular somatic spikes, which provide limited information about neural computation. Hence, it is necessary to record from neuronal dendrites, which can generate dendritic action potentials (DAPs) in vitro, which can profoundly influence neural computation and plasticity. We measured neocortical sub- and supra-threshold dendritic membrane potential (DMP) from putative distal-most dendrites using tetrodes in freely behaving rats over multiple days with a high degree of stability and sub-millisecond temporal resolution. DAP firing rates were several-fold larger than somatic rates. DAP rates were also modulated by subthreshold DMP fluctuations, which were far larger than DAP amplitude, indicating hybrid, analog-digital coding in the dendrites. Parietal DAP and DMP exhibited egocentric spatial maps comparable to pyramidal neurons. These results have important implications for neural coding and plasticity. Tetrodes are a bundle of four fine electrodes, commonly used for measuring somatic spikes from a distance, that is, extracellularly. Hence, they work well in freely behaving animals. However, tetrodes do not measure the membrane voltages of soma, let alone dendrites. Chronically implanted tetrodes also elicit a naturally occurring immune response, where glial cells encapsulate the tetrode and shield it from the extracellular medium. We tested the hypothesis that a segment of dendrite could get trapped between the tetrode tips before this glial encapsulation occurred (figure). This would enable us to measure the dendritic membrane voltage without penetrating it in freely behaving subjects.”

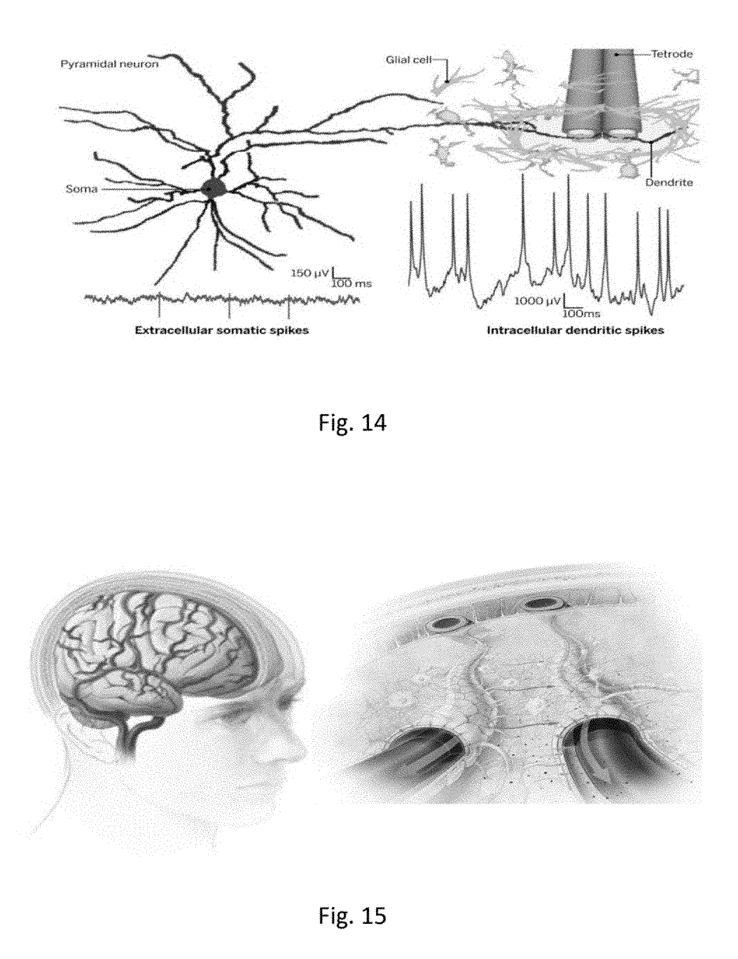

Referring to FIG. 14, “Dynamics of cortical dendritic membrane potential and spikes in freely behaving rats,” Jason J. Moore, Pascal M. Ravassard, David Ho, Lavanya Acharya, Ashley L. Kees Cliff Vuong, Mayank R. Mehta, Science 24 Mar. 2017:Vol. 355, Issue 6331, eaaj1497 DOI: 10.1126/science.aaj1497JJ Moore et al. Science 355 (6331). 2017 Mar. 9. This reference shows experimental evidence of action potential formation in dendrites. Moore's paper supports Glial cell's role in learning, at least as an abstraction.

The definition of glial cells set forth herein seems to be correct, since the brain tumor “glioma” the denominator of dendrite sum which has a potential singularity by division of zero. If the MFE of the brain is not correspondingly reduced, this singularity turns out to be pathological consistent with the medically known brain tumor “glioma.” The majority of brain tumors belong to this class of too-strong glue force. Notably, the former U.S. President Jimmy Carter suffered from glioma of three golf-ball sized large tumors. Nevertheless, the immunotherapeutic treatment using the newly marketed Phase-4 monoclonal antibody presenter drug (Protocol: 2 mg per kg body weight IV injection) that ID malignant cells and tag them for own anti-body to swallow the malignant cells made by Merck Inc. (NJ, USA) as Anti-Programming Death Drug-1 Keytruda (Pembrolizumab). Mr. Carter recovered in 3 weeks but it took 6 month to recuperate his own immune system (August 2015-February 2016).

Referring to FIG. 15: “Brain Drain” M. Nedergaad & S. Goldman, Sci. Am. March 2016.” 100B Astrocytes glials work day and night to clean out the energy production junks such as Omega Amyloid in order to keep up 20% energy usage of the whole human body.

New York Times (Pam Belluck, Nov. 23, 2016). An experimental Alzheimer's drug that had previously appeared to show promise in slowing the deterioration of thinking and memory failed in a large Eli Lilly clinical trial, dealing a significant disappointment to patients hoping for a treatment that would alleviate their symptoms. The failure of the drug, solanezumab, underscores the difficulty of treating people who show even mild dementia, and supports the idea that by that time, the damage in their brains may already be too extensive. And because the drug attacked the Amyloid plaques that are the hallmark of Alzheimer's, the trial results renew questions about a leading theory of the disease, which contends that it is largely caused by Amyloid buildup.

Astrocytes are closely related to blood vessels and synapses. In fact, they have processes that are in direct contact with both blood vessels and synapses. This makes them ideal candidates for neurovascular regulation. In 2003, an increase in the amount of intracellular Ca²⁺ in astrocytic endfeet was discovered upon electrical stimulation of neuronal processes. The increase led to dilatation of local cerebral arterioles, successfully linking astrocytes with a role in neurovascular regulation. But an increase in astrocytic Ca²⁺ is not only mobilized by neuronal activation. A number of transmitters, neuromodulators and hormones can in fact do the exact same thing, independently of synaptic transmission in neurons. Therefore, astrocytes also regulate the response of the cerebral vasculature. Further still, studies have shown that astrocytes could also account for a significant portion of energy consumption in the brain (see references 2 and 3). Although, neurons obtain most of their energy by glycolysis, astrocytes derive much energy from oxidative metabolism and the associated release of glial transmitters, such as ATP, during Ca²⁺ signaling. Khalil A. Cassimally Jul. 17, 2011: “Are fMRI Telling The Truth? Role of Astrocytes in Cerebral Blood Flow Regulation” in terms of Astrocytes glial cells driven by MFE that will appear in medical image processing elsewhere.

This approach of medical imaging early at the glymphatic system (M. Nedergaad & S. Goldman (“Brain Drain Sci. Am. March 2016”)

H_{brain}=Eₒ+\vec{g}ᵢ·[W_{i,j}](\vec{S}ⱼₒ−[Wⱼₖ]\vec{X}ₖ)+k_{B}TₒΣSᵢ log Sᵢ+(λ₀−k_{B}Tₒ)(ΣSᵢ−1) (25)

This MFE of the brain Internal Energy E can be Taylor expanded in terms of input brain imaging intensity \vec{X}ₖ , then it can determine MFE by imaging as the negative slope as the glial cells behavior.

### Conclusion

The work of others supports the unified theory of all neuroglia cells. This might be deja vu of the days when McCullough-Pitts and John Von Neumann defined the neuron. It has helped engineers and biologists to fuse both sides of knowledge to make advancements. It is believed that once the concept of house-keeping neuroglia cells has been identified mathematically, potential application areas could be wide open and leave only to the imagination with all innovative readers. Some are suggestive, and by no means to pre-empt the topic as follows:

(1) The biomedical industry can apply ANN & SDL to these kinds of profitable BDA, namely Data Mining (DM) in Drug Discovery, for example, Merck Anti-Programming Death for Cancer Typing beyond the current protocol (2 mg/kg of BW with IV injection), as well as NIH Human Genome Program, or EU Human Epi-genome Program BDA Drug Discovery: FDA Application of Explainable Computational Intelligence.

Is the Herbal Mushroom G Lucidum, Lingzhi (that 2000 Nobel Laureate Literature Mr. Gao Xingjian recovered in cancer) similar to Merck immunotherapy Keytruda (Pembrolizumab) drug (that President Jimmy Carter Liver and Brain Metastasis cancer: August 2015 ˜February 2016)? Merck drug (yellow balls) are targeted at the Programmed cell Death 1 (PD-1) receptor and allows the body's own immune system go after the cancer cells. While they are all worked on human immune systems, the key difference between Eastern Herbal Medicine and Western Molecular personalized precision targeted drug is mainly in that the holistic is slow in nature of herbal drug for years versus fast drug in half a year.

(2) SDL & ANNs should be applied to enhance the Augmented Reality (AR) & Virtual Reality (VR), etc. for CI to aid the Training purpose, similar to proactive chess game playing.

(3) There remains BDA in the law & order societal affairs, for example, flaw in banking stock markets, and law enforcement agencies, police and military forces, who may someday require the “chess playing proactive anticipation intelligence” to thwart the perpetrators or to spot the adversary in a “See-No-See” Simulation & Modeling, at the man-made situation, for example, inside-traders; or in natural environments, for example, weather and turbulence conditions.

(4) Furthermore, BDA is divided into open sets of Large Data Analysis (LDA) defined as the relational data basis (Attribute, Object, Value)=(Color, Apple/McIntosh, Red Delicious/Green Tarnish) or the other homogeneous data structure (SS#, Name, Sex, Age, Profession, etc.). Some of them may require a NI effortless decision making known as Unsupervised Deep Learning (UDL) given, therefore, we have developed from thermodynamics for the first time as follows.

(5) Explainable A: One can help DARPA (I2O) during PPI apply the Supervised Deep Learning Classifier vs. Unsupervised Deep Learning for Ortho-Normal Salient Feature Extraction

What is the Cost Functions for supervised and unsupervised DL? Supervised DL utilizes the LMS errors for AI, ANN learnable relational databases; Unsupervised DL utilizes the Minimum Free Energy (MFE) at BNN at Helmholtz MFE for Natural Intelligence (NI), if and only if (i) Isothermal Brain (ii) Power of Pairs for BNN Learning

[W(i,j)]X(in, pair)(t)=S(out, fusion)(t)

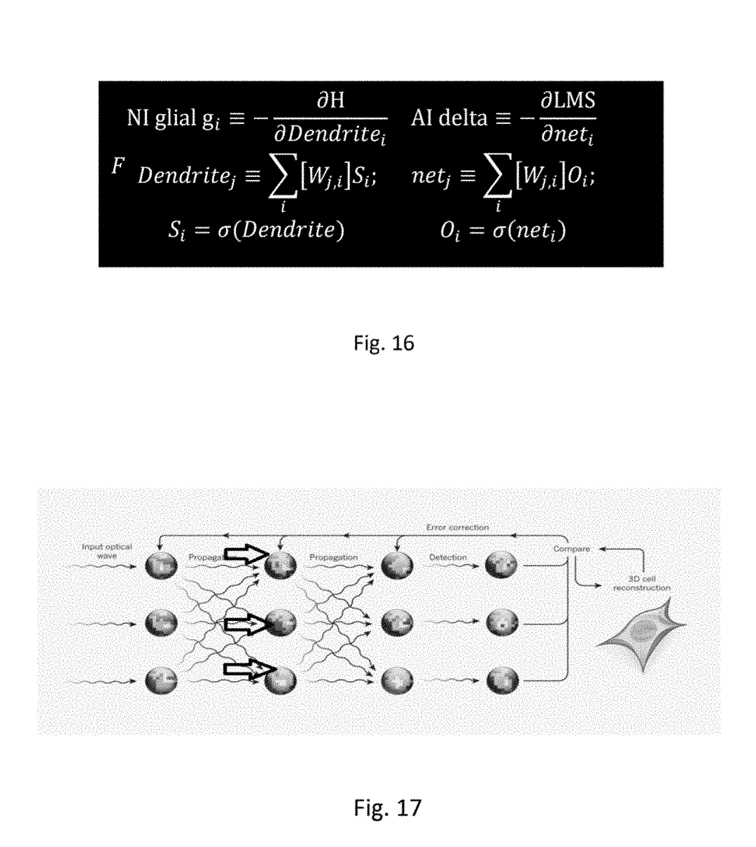

As suggested in FIG. 16, this set of relationships is the new Rosetta Stone that relates classical ANN with modern BNN.

FIG. 17 shows deep learning back-prop mediated through glial cells in BNN.

### REFERENCES

1. Mary Nedergaad, Steve Goldman (Sweden & Rochester) “Brain Drain,” Sci. Am. March 2016, pp. 45-49.
2. Nobel Prize in Medicine & Physiology has been given in 2012 to the discovery genes by Kyoto Prof. Shinya Yamanaka, and these 4 Yamanaka genes can be unwind cells back to the embryonic (adult cells induced pluripotent: mice, Dolly Sheep, Homo sapiens longevity)
3. Common Sense Longevity: Sleep Tight, Eat Right (Matterson. Calorie Restriction: Luigi Fontana Alternative Fasting, Wash U.), Deep Exercise (e.g. Tai Chi Quan), Be Happy (Vegas: Yoga).
4. “Learning Machine,” Nicola Jones V. 505, pp 146-148, 2014;
5. “Deep Learning,” Yann LeCun, Yoshui Bengio, Geoffrey Hinton, V. 521, pp. 436-440, 2015.
6. “Natural Intelligence Neuromorphic Engineering,” Harold Szu, Elsevier 2017, pp. 1-350.
7. “ANN, Deep Learning & Apps,” Harold Szu, Henry Chu & Simon Foo (Gulf Mexico Spring School Apr. 16-19, 2017 Tallahassee Fla., Elsevier Book Publisher)
8. “Unsupervised Learning at MFE” (single layer LCNN for one class breast cancer or not), appeared in Harold Szu, Lidan Miao, Hairong Qi, Proc. SPIE Vol. 6576, p. 657605, (2007)
9. Multiple Layer Deep Learning appeared in “Introduction to Computing with Neural Nets,” Richard Lipmann, IEEE ASSP Magazine April 1987 & PDP MIT book (David Rumelhart, James McCelland); Paul Werbos Thesis.
10. Harold Szu, Binh Tran, Francois Lalonde, “Noninvasive detection of brain order-disorder transitions using functional f-EEG” 28 May 2014, SPIE Newsroom. (DOI: 1117/2.1201405.005446)
11. “Deep learning ANN & Appl.” book edited by Foo, Chu, Szu et al. from GMSS Tallahassee Fla. Apr. 16-18, 2018 Elsevier 2018.

I claim:

1. A method of diagnosing a disorder, comprising:
obtaining a medical image of a subject;
computing a Helmholtz Minimum Free Energy from the medical image;
determining a negative slope of the Helmholtz Minimum Free Energy;
computing a glial force from the negative slope; and
diagnosing the existence of a disorder in the subject if a value of the glial force is within a predetermined range.

2. The method of claim 1, wherein the disorder is a brain disorder.

3. The method of claim 2, wherein the disorder is Alzheimer's disease.

4. The method of claim 2, wherein the disorder is Parkinson's disease.

5. The method of claim 2, wherein the disorder is schizophrenia.

6. The method of claim 2, wherein the disorder is multiple sclerosis.

7. The method of claim 1, wherein the disorder is epilepsy.

8. The method of claim 1, wherein the disorder is rheumatoid arthritis.

9. The method of claim 1, wherein the subject is a human subject.
